(19) United States
(12) Patent Application Publication
(10) Pub. No.: US 2022/0079981 A1
(43) Pub. Date: Mar. 17, 2022

(54) LAVAGE TECHNIQUES AND VIRUCIDAL COMPOSITIONS COMPRISING HYPOCHLOROUS SOLUTIONS

(71) Applicant: FaceRestoration Ltd, Norfolk (GB)

(72) Inventors: Rachna Murthy, Norfolk (GB); Jonathan Roos, Norfolk (GB)

(73) Assignee: FaceRestoration Ltd

(21) Appl. No.: 17/475,741

(22) Filed: Sep. 15, 2021

Related U.S. Application Data
(60) Provisional application No. 63/078,786, filed on Sep. 15, 2020.

(51) Int. Cl. A61K 33/20; A61K 9/08; A61K 9/00; A61P 31/02

(52) U.S. Cl. CPC A61K 33/20; A61P 31/02; A61K 9/0048; A61K 9/08

(57) ABSTRACT

The present disclosure provides methods and compositions for treating and preventing conditions characterised by infection and/or inflammation of mucus membranes, skin and surrounding structures, especially of the eyes, ears & ear canal, nose, pharynx, mouth, urethra, vagina, glans penis, anus, skin and nipples. The presently disclosed methods may include administering hypochlorous acid to the patient's surfaces, such as in the form of a composition described within this manuscript. In many embodiments, the disclosed compositions may have varying pH and/or concentrations of hypochlorous, for example specific pH and hypochlorous concentrations that may be tailored for each treatment environment. In many embodiments, the disclosed compositions may be administered with site specific applicators, configured so as to increase tolerability.

### FIELD

The present disclosure is directed to methods and compositions for treating and preventing infection and/or inflammation of mucus membranes, skin, and surrounding structures, especially of the eyes, in patients in need thereof.

### CROSS-REFERENCE TO RELATED APPLICATIONS

This application claims benefit of priority pursuant to 35 U.S.C. § 119(e) of U.S. provisional patent application No. 63/078,786 entitled “LAVAGE TECHNIQUES AND VIRUCIDAL COMPOSITIONS COMPRISING HYPOCHLOROUS SOLUTIONS,” filed on 15 Sep. 2020, which is hereby incorporated by reference in its entirety.

### BACKGROUND

There are multiple inflammatory and infectious conditions, and inflammation triggered by infectious conditions associated with mucus membranes and the junction with internal orifices and the external skin and its appendages. This includes the ocular surface, ear, nose, mouth, nipple, vagina, glans penis, urethra and the anus in adults and children. These conditions are often treated with empirical antibiotics, antivirals, antifungals and anti-inflammatory agents depending or the suspected aetiology.

Such treatments are limited by microbial resistance, irritation, hypersensitivity, cytotoxicity, staining and drug toxicity depending on the treatment employed. Not all are suitable therefore for use in childhood or during pregnancy or breastfeeding, or on the ocular surface.

There is a need therefore for an effective antimicrobial agent that does not result in resistance, whilst also avoiding side effects of toxicity and irritation.

### SUMMARY

Disclosed herein are methods and systems for treating a condition associated with a viral infection comprising: a solvent; hypochlorous, wherein the active free chlorine (AFC) of hypochlorous may be between about 10 and 300 ppm, and the pH of the composition is between about 4 and 8. In some embodiments the virus may be SARS, for example SARS Coronavirus-19, and/or the pH of the composition is from about 6.5 to about 7.5. In many embodiments the condition may be selected from ocular, peri-ocular, nasal, ear, mouth, genitourinary, mastitis, nipple infection, eczema, viral rash, acne, hidradenitis suppurativa, wounds, incisions, diaper rash, blepharitis, canaliculitis and dacryocystitis, endophthalmitis, herpes zoster ophthalmicus, rhinitis, nasal staphylococcus, otitis media, otitis externa, swimmer's ear, pharyngitis, stomatitis, vaginitis, urethritis, balanitis, external and internal hordeolum, chalazion, and conjunctivitis, and/or the composition may be a liquid, spray, ointment, or other topical treatment, and/or the AFC may be from about 70 to 200 ppm.

Also disclosed are compositions for treating a condition associated with an infection comprising: a solvent; hypochlorous, wherein the concentration of active free chlorine (AFC) may be between about 10 and 300 ppm, and the pH of the composition is between about 4 and 8, for example 70-200 ppm and pH 6.5 to about 7.5. In many embodiments, the condition may be selected from ocular, peri-ocular, nasal, ear, mouth, genitourinary, mastitis, nipple infection, eczema, viral rash, acne, hidradenitis suppurativa, wounds, incisions, diaper rash, blepharitis, canaliculitis and dacryocystitis, endophthalmitis, herpes zoster ophthalmicus, rhinitis, nasal staphylococcus, otitis media, otitis externa, swimmer's ear, pharyngitis, stomatitis, vaginitis, urethritis, balanitis, external and internal hordeolum, chalazion, and conjunctivitis, and/or the composition may be a liquid, spray, ointment, or other topical treatment. In many embodiments, the infection may be caused by bacterial, spores, fungi and viruses including, for example, without limitation, Apergillus niger, Candida albicans, Corynebacterium amycolatum, Enterobacter aerogenes, Escherichia coli, Haemophilus influenza, Klebsiella pneumoniae, Methicillin-resistant Staphylococcus aureus, Micrococcus luteus, Proteus mirabilis, Pseudomonas aeruginosa, Serratia marcescens, Staphylococcus aureus, Staphylococcus epidermidis, Staphylococcus haemolyticus, Staphylococcus hominis, Staphylococcus saprophyticus, Streptococcus pyogenes, Vancomycin-resistant Enterococcus faecium and viruses including Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae, for example SARS and MERS Coronavirus, low pathogenic avian influenza virus (AIV), H7N1, for example SARS Coronavirus-19.

Also disclosed are methods of treating or preventing a viral infection comprising: dispensing a sufficient amount of a composition comprising hypochlorous toward an eyeball of a subject; allowing the composition to contact the eyeball; thereby treating or preventing a viral infection, wherein the concentration of active free chlorine (AFC) in the composition may be between about 10 and 300 ppm, for example about 70-200 ppm, or 80 ppm. In many embodiments, the virus may be selected from Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae, for example SARS and MERS Coronavirus, low pathogenic avian influenza virus (AIV), H7N1, and COVID-19, for example SARS CoV-2. In many embodiments, the pH may be about 6.5 to 7.6.

### BRIEF DESCRIPTION OF THE DRAWINGS

FIG. 1 is a graphical representation of the effect of pH on relative abundance of the various hypochlorous species.

FIG. 2 shows results for a representative subject treated with the disclosed composition.

### DETAILED DESCRIPTION

The present compositions and methods are useful for treating various diseases and conditions involving infection and/or inflammation. For example, the disclosed methods and compositions are useful in treatment of mucus membranes and surrounding structures including the conjunctiva, eyelid skin, eyelashes, eyebrows, eyelid glands, and lacrimal drainage including canaliculi and nasolacrimal: bacterial and viral conjunctivitis blepharitis, external and internal hordeolum, chalazion, canaliculitis and dacryocystitis.

The disclosed methods and compositions may also provide a prevention of infection and pre-treatment for intraocular surgery and ocular and peri-ocular interventions and contact lens wear.

Conditions that may be treated with the disclosed compositions and methods also include various conditions affecting the mucocutaneous junction of the ear (including inner ear, middle ear, ear canal, and ear drum), nose, and mouth, including: rhinitis, sinusitis, rhinorrhea, otitis media, otitis externa, stomatitis, mouth ulcers and dental implant care.

Conditions that may be treated with the disclosed compositions and methods include infection and/or inflammation involving mucocutaneous junctions of the urethra, vagina, anus and, also where mucocutaneous contact occurs such as with breast feeding.

The disclosed methods may include administering a composition of hypochlorous acid to the affected areas described, by administration with drop method or spray for the required mucus membrane indication.

The disclosed methods may include providing an effective method for cleansing and treating the inflamed and/or infected regions, in a manner that is free of, or significantly free of toxicity, hypersensitivity, staining, and drug resistance.

The disclosed methods may include administration of a pure, pH balanced, buffered solution for delivery to mucus membranes of the ocular surface, mouth, nose, ear, vagina, urethra and anus.

The disclosed methods and compositions may retain stability and effective antimicrobial activity with available free chlorine over 50 ppm (for example in the range of about 80-300 ppm).

The disclosed methods and compositions may be useful as an alternative or adjunct therapy to conventional topical antibiotics, antivirals, decongestants, antihistamines, and steroid treatments both in and outside the hospital environment as well as on the go including on aircraft and other modes of public transport. The disclosed methods and compositions may be non-sensitising so can be used without risk to others. The disclosed methods and compositions may allow a means of antisepsis prior to examination of or intervention to mucus membranes.

The disclosed methods and compositions may provide a composition containing hypochlorous acid for treating inflamed and/or infected regions of the eye, ear, nose (including sinuses), mouth or throat, vagina, glans penis and perineum. In most embodiments, the composition is an electrolysed saline solution comprising highly pure hypochlorous acid with a pH that is consistent and compatible with the ocular surface and/or mucus membranes—for example, in a range of about 4 to about 7, and an available free chlorine (AFC) acid solution content of from about 80 to about 300 parts per million (ppm).

### Purity:

The disclosed compositions may retain purity with minimal sodium hypochlorite for stability. In most embodiments, a pure composition may be substantially free of organic material and have a concentration of hypochlorous that is substantially greater than that of sodium hypochlorite.

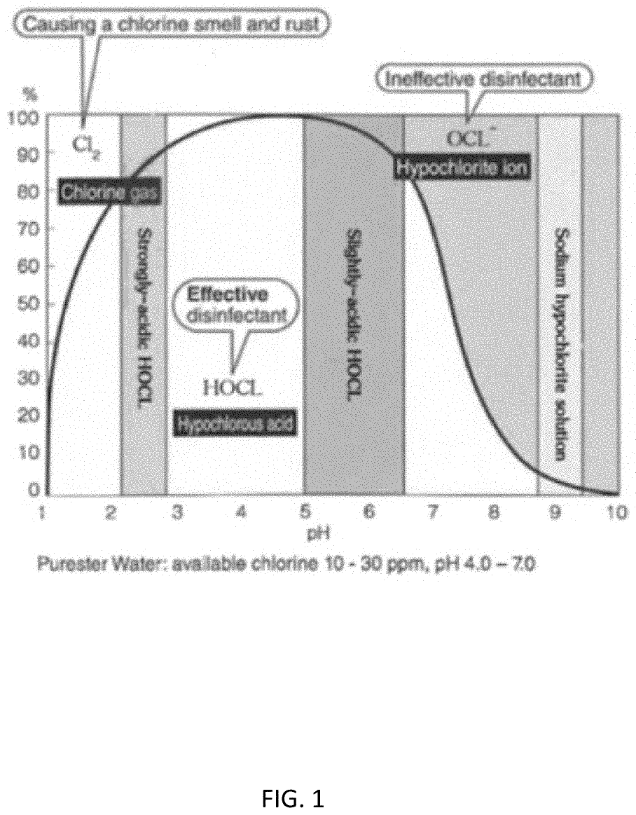

Hypochlorous has no charge and has low molecular weight. These characteristics render hypochlorous better able to penetrate cell walls than sodium hypochlorite, and, generally, a more effective disinfectant. Hypochlorous is a weak acid that dissociates into hypochlorite ion (OCl—) and hydrogen ion according to the equation HOCl->H++OCl— (hypochlorite ion). These three species exist in an equilibrium which is pH and temperature dependent; the sum of these is referred to as the total available chlorine. At 25° C. and pH 7.5 half of the total chlorine is present as HOCl and the other half as OCl—. The dissociated hypochlorite ion (OCl—) predominates at higher pH values, above 7.5, while the undissociated hypochlorous acid (HOCl) predominates at lower pH values. For example, from pH 4.0 to 7.0 the HOCl species predominates. As HOCl is the main disinfecting agent, compositions in this pH range may be useful in treating various disorders although the concentration of chlorine may be relatively low, for example about 10-30 ppm. FIG. 1 is a graphical representation of the effect of pH on relative abundance of the various hypochlorous species.

Sodium hypochlorite is negatively charged and may be electrostatically repelled by cell walls. In many embodiments compositions lacking sodium hypochlorite may be unstable. In many embodiments, compositions comprising sodium hypochlorite may contain around 5% hypochlorous acid—in these cases a higher concentration of chlorine, in the range of about 100-200 ppm, may be useful, where the remaining 95% is in the OCl—, which in many cases has a lower bacteriocidal/virucidal effect. During manufacture, this may remain unused and may be discarded as waste water.

### Preservation:

The disclosed compositions may have a shelf life of 18 months-2 years, wherein the concentration may decrease by less than half over about two years.

pH:

The disclosed compositions may possess a pH that allows compatibility with the mucus membrane indication whilst also maximising antimicrobial activities and minimising undesirable degradation of the product. Concentrations of AFC over about 50 ppm are, in most embodiments, required for effective antimicrobial and virucidal properties. In many embodiments, the pH may be greater than about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.1, 8.2, 8.3, 8.4, or 8.5 and less than about 8.6, 8.5, 8.4, 8.3, 8.2, 8.1, 8.0, 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, 4.5, 4.4, 4.3, 4.2, 4.1, 3.9, 3.8, 3.7, or 3.6. As used herein, the term about may refer to a variance of +/−10% of the value provided, for example “about 10.0” may range from 9.0 to 11.0.

### Indication:

The disclosed composition is broadly effective for cleansing, disinfecting, and/or reducing inflammation of the eyes, ears, nose, mouth, and/or throat, nipple, vagina, glans penis and perineum. The disclosed composition may be useful as a pre-intervention cleanser, pre-operative cleanser and as an alternative or adjunct to conventional treatments, and is particularly suitable for prolonged use and hygiene, especially for individuals prone to such infections and/or inflammatory conditions, or individuals that typically experience hypersensitivity with other treatments.

### Activity:

The disclosed composition may have broad spectrum activity to a variety of bacterial, spores, fungi and viruses including, for example, without limitation, Apergillus niger, Candida albicans, Corynebacterium amycolatum, Enterobacter aerogenes, Escherichia coli, Haemophilus influenza, Klebsiella pneumoniae, Methicillin-resistant Staphylococcus aureus, Micrococcus luteus, Proteus mirabilis, Pseudomonas aeruginosa, Serratia marcescens, Staphylococcus aureus, Staphylococcus epidermidis, Staphylococcus haemolyticus, Staphylococcus hominis, Staphylococcus saprophyticus, Streptococcus pyogenes, Vancomycin-resistant Enterococcus faecium and viruses including Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae, for example SARS and MERS Coronavirus, low pathogenic avian influenza virus (AIV), H7N1. Coronavirus-19 may refer to SARS-CoV-2 as well as any variant thereof.

Table I provides a non-limiting and non-exhaustive list of indications that may be treated with the presently disclosed methods and compositions. The table also provides one embodiment of a composition and method that may be used in treating the condition, for example pH, AFC, carriers, additives, dispensing methods, etc.

Table I lists indications, modes of delivery and compositions as disclosed herein. Dosing of od (once per day), bd (twice per day), tds (three times per day) as required, or more. For long term use. pH buffered solution of Phosphoric Acid and Sodium Hypochlorite 0.01-0.03%.

| | | Dispensing | Composition | Other |
|---|---|---|---|---|
| Indication | pH | method | (AFC) | characteristic |
| Ocular | 7.0-7.3 | Drop, Mist, | 80 ppm | Carrier: liquid |
| | | Spray, | | paraffin for |
| | | Ointment | | ointment. Buffer |
| | | | | phosphoric acid |
| Nasal | 5.5-8.3 | Drop, Spray, | 80 ppm | Carrier: Liquid |
| | | Ointment, | | paraffin for |
| | | lavage | | ointment. Buffer |
| | | | | phosphoric acid |
| Oral | 6.2-7.3 | Solution, | 80 ppm | Buffer phosphoric |
| | | Spray, lavage, | | acid |
| | | mouthwash | | |
| Vagina | 3.8-4.5 | Spray, Mist, | 100 ppm- | Buffer phosphoric |
| | | Lavage | 300 ppm | acid |
| Penis | 7.1-8 | Spray, Mist, | 80 ppm | Buffer phosphoric |
| | | Lavage | | acid |
| Ear | 5.0-7.8 | Drop, Spray | 100 ppm | Buffer phosphoric |
| | | | | acid |
| Skin | 4.7-5.75 | Spray, Mist, | 100 ppm- | Buffer phosphoric |
| | | Lavage | 300 ppm | acid |
| Contact | 7.0 | Spray, Mist, | 80 ppm | Buffer phosphoric |
| lens/dental | | Solution | | acid |
| retainer/ | | | | |
| implant | | | | |

Disclosed herein are methods and compositions for treatment of conditions associated with infection and/or inflammation of mucus membranes and surrounding structures. As used herein, “a sufficient amount” means an amount of the composition applied to, used or worn by the subject at the treatment site to provide relief, improve the appearance, or reduce the concentration of infective agents in the area.

### Scope

The present disclosure relates to highly purified compositions and methods of using same, for treating or preventing conditions characterised by infection and/or inflammation. In many embodiments, the disclosed methods may comprising administering a composition comprising hypochlorous, for example a solution of pure hypochlorous acid, to an affected treatment area. The disclosed compositions may be administered to the affected area and/or susceptible areas that may include or be positioned near mucus membranes. The disclosure is useful in treating and preventing conditions affecting various locations on and in the mammalian body, for example the eye, ear, nose, mouth, throat, nipple, vagina, glans penis, urethra, skin, and or/peri-anal area. In many embodiments, the disclosed methods and compositions may be useful in the care of babies hygiene, nappy rash, and to aid breast-feeding by cleaning nipples and surfaces.

The disclosed compositions are portable and, in many cases, may be safely administered by the patient and/or a medical provider. Such an on-the-go multipurpose solution that is safe for sensitive areas, including the ocular surface, is a novel development in the treatment and prevention of primary bacterial and viral infections, secondary infections and inflammation.

The disclosed compositions and methods are useful in treating a variety of conditions related to infection and/or inflammation at or near various surfaces, including mucus membranes. In various embodiments, the disclosed surfaces for treatment may be at or near the eyes, nose, ears, mouth, face, groin, arm pit, breasts, anus, penis, or vagina.

Patients that may be treated with the disclosed methods and compositions include adults, children, and infants. In many embodiments, the patient is an adult in need of treatment to reduce or prevent infection or inflammation due to infection. In many embodiments, the patient is an adult male or adult female. In some embodiments, the patient may be a nursing female with need for a treatment to reduce infection of a nipple prior to, during, or after breast feeding. In many embodiments, the disclosed infants may have an infection related to wearing a diaper or nappy. In many embodiments, an infant may be less than the age of about 2 or 3 years, a child may be from about 2 or 3 years to about 10 or 11 years of age, and an adult may have an age greater than about 10 years.

The disclosed compositions comprise hypochlorous acid, or hypochlorous, at varying concentration, and have a pH between about 3 and 8, for example pH 4-7. Hypochlorous may refer to a compound of the structure HOCl, which, in most cases, is in rapid equilibrium with —OCl and +H. In many embodiments, the concentration of hypochlorous is determined by measuring the concentration, in parts per million (PPM or ppm), of hypochlorite, —OCl, which may be referred to as active free chlorine (AFC). The concentration of hypochlorous may also be measured as a percentage of sodium hypochlorite, NaOCl, where a 1% solution comprises about 1 g NaOCl in 100 mL of H₂O. In most embodiments, the disclosed compositions may comprise between about 20 and 300 ppm or about 0.002 and 0.03% AFC. In many embodiments, the % AFC may be greater than about 0.0005, 0.0010, 0.0015, 0.0020, 0.0025, 0.0030, 0.0035, 0.0040, 0.0045, 0.0050, 0.0055, 0.0060, 0.0065, 0.0070, 0.0075, 0.0080, 0.0085, 0.0090, 0.0095, 0.0100, 0.0120, 0.0140, 0.0160, 0.0180, 0.0200, 0.0220, 0.0240, 0.0260, 0.0280, 0.0300, 0.0320, 0.0340, 0.0360, 0.0380, or 0.0400% AFC and less than about 0.0400, 0.0380, 0.0360, 0.0340, 0.0320, 0.0300, 0.0280, 0.0260, 0.0240, 0.0220, 0.0200, 0.0180, 0.0160, 0.0140, 0.0120, 0.0100, 0.0095, 0.0090, 0.0085, 0.0080, 0.0075, 0.0070, 0.0065, 0.0060, 0.0055, 0.0050, 0.0045, 0.0040, 0.0035, 0.0030, 0.0025, 0.0020, 0.0015, 0.0010% AFC.

The AFC ppm of the disclosed compositions may vary according to the indication. In most embodiments, especially for treatment of or near mucus membranes or sensitive skin, the composition may comprise between about 60 and about 120 ppm AFC, for example from about 80 to about 100 ppm. In some embodiments, the AFC may be about 80 ppm. In some embodiments, the AFC may be about 100 ppm. In most embodiments, where the composition is applied to skin at or near the groin, nipple, or armpit, the solution may be greater than about 100 ppm AFC, for example about 100 to about 300 ppm. In many embodiments, the AFC may be greater than about 25 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, or 375 ppm and less than about 375, 370, 365, 360, 355, 350, 345, 340, 335, 330, 325, 320, 315, 310, 305, 300, 295, 290, 285, 280, 275, 270, 265, 260, 255, 250, 245, 240, 235, 230, 225, 220, 215, 210, 205, 200, 195, 190, 185, 180, 175, 170, 165, 160, 155, 150, 145, 140, 135, 130, 125, 120, 115, 110, 105, 100, 95, 90, 85, 80, 75, 70, 65, 60, 55, 50, 45, 40, 35, or 30 ppm.

The disclosed hypochlorous solutions may be combined with various compounds/ingredients to create a suitable vehicle for administration. In some embodiments, the solution may be administered as a lavage, irrigation, droplet, spray, mist, etc. In some embodiments, the hypochlorous solution may be administered to the treatment area as an ointment or cream, for example a paraffin-based ointment. Topical formulations according to the present invention may take the form of compositions suitable for topical application to the body surface, and may comprise, for example, a cream, lotion, solution (e.g., liquid spray), gel, film, ointment, paste, plaster, paint, bioadhesive, or the like.

Solvents, including relatively small amounts of water or alcohol, may be used to solubilize certain formulation components. Suitable solvents include glycerin, SD 40 alcohol, lecithin, poloxamers, Miglyol and methyl pyrrolidone, or combinations thereof. Other suitable solvents include glycerol, diluted alcohol isopropanol, hexylene glycol and propylene glycol. Menthol may be also be added to the topical formulation of the present invention. In one embodiment, surfactant(s) may be employed in the topical formulation according to the present invention, for example, in an amount of from about 0.05% to about 5.0% w/w. Suitable surfactants may include, but are not limited to sodium lauryl sulfate, benzalkonium chloride, cetylpyridinium chloride, sodium laurate, cetyltrimethylammonium bromide, Poloxamer (231, 182, 184), Tween (20, 40, 60, 80) and combinations thereof.

The disclosed volumes of solution may range from about 5 μl to about 50 ml. In some embodiments, the volume may be less than about 50 ml, 40 ml, 30 ml, 20 ml, 10 ml, 9 ml, 8 ml, 7 ml, 6 ml, 5 ml, 4 ml, 3 ml, 2 ml, 1 ml, 950 μl, 900 μl, 850 μl, 800 μl, 750 μl, 700 μl, 650 μl, 600 μl, 550 μl, 500 μl, 450 μl, 400 μl, 350 μl, 300 μl, 250 μl, 200 μl, 150 μl, 100 μl, 90 μl, 80 μl, 70 μl, 60 μl, 50 μl, 40 μl, 30 μl, 20 μl, 10 μl, 9 μl, 8 μl, 7 μl, or 6 μl and more than about 5 μl, 6 μl, 7 μl, 8 μl, 9 μl, 10 μl, 20 μl, 30 μl, 40 μl, 50 μl, 60 μl, 70 μl, 80 μl, 90 μl, 100 μl, 150 μl, 200 μl, 250 μl, 300 μl, 350 μl, 400 μl, 450 μl, 500 μl, 550 μl, 600 μl, 650 μl, 700 μl, 750 μl, 800 μl, 850 μl, 900 μl, 950 μl, 2 ml, 3 ml, 4 ml, 5 ml, 6 ml, 7 ml, 8 ml, 9 ml, 10 ml, 20 ml, 30 ml, and 40 ml.

The disclosed compositions may be administered in various dosage regimen. In many embodiments, the disclosed composition may be administered as needed, once per day (od), twice per day (bd), three times per day (tds), or more. In many embodiments, an individual application may be one or more drops or sprays, wherein the drop may be less than about 70 μl, 60 μl, 50 μl, 40 μl, 30 μl, 20 μl, or 10 μl and more than about 5 μl, 10 μl, 20 μl, 30 μl, 40 μl, 50 μl, or 60 μl and the spray may be greater than about 50 μl, 60 μl, 70 μl, 80 μl, 90 μl, 100 μl, 150 μl, or 200 μl, and less than about 250 μl, 200 μl, 150 μl, 100 μl, 90 μl, 80 μl, 70 μl, or 60 μl per pump.

Below is a non-exhaustive list of conditions that may be treated with the disclosed compositions and methods.

### Conditions:

### 1. Ocular and Peri-Ocular Conditions

a. Conjunctivitis in Neonates and Adults & Trachoma

Conjunctivitis is the inflammation and/or infection of the mucus membrane lining of the eye, from overgrowth of commensals or transmission from contact, faeco-oral spread or airborne contaminants. Common bacterial organisms responsible for conjunctivitis are Staphylococcus and Streptococcus species, Neisseria or Chlamydia which may be transferred vertically from an infected mother to the neonate from the vagina and, also is a global issue resulting in Trachoma in the developing world, particularly sub-Saharan Africa. Viral conjunctivitis is usually due to adenovirus transmitted via respiratory droplets and contact. Coronavirus is of particular relevance due to SARS Coronavirus-19 which is also airborne in transmission, but can result in significant morbidity and mortality from infection. Conjunctivitis secondary to infection is contagious and can easily be spread from person to person. Contact lenses of extended wear can harbour microbes and be a fomite for spread of infection to the ocular surface.

Conjunctivitis un-treated can result in sequelae including corneal ulceration, misdirection of eyelashes, ocular irritation and abrasion of the cornea, pain and loss of vision secondary to scarring.

Current treatments for bacterial conjunctivitis are empirical with antibiotics. Whilst bacterial cultures can be obtained to guide treatment, these are often negative, and antibiotic long term use can result in drug resistance.

Treatment of viral conjunctivitis is largely symptom control and allowing the disease to run its course. Antiviral treatments are not commonly used for viral conjunctivitis due to drug side effects, topical steroid drops are used in severe cases to limit the inflammation secondary to viral infection and scarring.

### CompositβIon and Delivery for Ocular Use:

Eye pH compatible buffered hypochlorous solution with concentration of 0.008% sodium hypochlorite or 80 ppm available free chlorine (AFC).

For daily dosing bd-tds and long term use.

Dispensed as drop for ocular surface, or mist for ocular surface and periocular use, or ointment with liquid paraffin base.

b. Blepharitis

Blepharitis is inflammation of the eyelid margin, including the mucocutaneous junction, the eyelash and accessory glands in the eyelids. The most common cause is due to staphylococcal hypersensitivity and overgrowth. Staphylococcus aureus is the most common implicated organism. Long term untreated blepharitis can result in chronic ocular irritation and dry eye disease. Treatment involves lid hygiene and usually the empirical use of topical antibiotic and/or steroid. Blepharitis can lead to obstruction of the eyelid glands and result in a chalazion (blocked Meibomian gland) or stye (blocked gland or Zeis; such as from a bacterial infection, and may present as a red, painful lump near the edge of your eyelid that may look like a boil or a pimple. In some embodiments, sties may contain pus). Secondary infections resulting from blockage of the eyelid glands can result in cellulitis of the skin in the periocular area and worst case scenario orbital cellulitis by spreading behind the orbital septum, this can risk loss of vision.

Early treatment of a chalazion with correctly applied massage, eyelid hygiene and control of blepharitis, can avoid a large irritable cyst, which can be unsightly, blur the vision and in a developing child under the age of 8 result in permanent damage to the vision.

When lid massage is delayed or unsuccessful, surgical incision and drainage/curettage of the cyst, or intra-lesional injection of steroid is employed.

Composition and Delivery for eyelid use:

Eye pH compatible buffered hypochlorous solution with concentration of 0.008%-0.01% sodium hypochlorite or 80 ppm-100 ppm available free chlorine (AFC).

For daily dosing bd-tds and long term use.

Dispensed as spray or mist for ocular surface and periocular use, or ointment.

c. Canaliculitis and Dacryocystitis

The canalicular drainage channels and lacrimal sac and duct are the conduits for tears from the eye surface to the nasal cavity. Blepahritis results in narrowing of the drainage orifice, the punctum and canalicular systems, narrowing in the nasolacrimal duct results in stasis of tears in the lacrimal sac and can result in, not just chronic watery eyes but also inflammation and infection in the form of dacryocystitis. Dacryocystitis causes chronic mucoid discharge which blurs vision, swelling in the inner corner of the eye and can result in cellulitis.

Canaliculitis can involve Actinomyces infection and further obstruct tear drainage due to a granular exudate. Treatment involves irrigating the canaliculus with penicillin and laying open or marsupialisation of the canaliculus. Dacryocystitis is usually secondary to streptococci or Staphylococcus aureus and is usually treated with antibiotics and ultimately surgery to clear the nasolacrimal duct obstruction.

Composition and Delivery for lacrimal drainage use:

Eye pH compatible buffered hypochlorous solution with concentration of 0.008% sodium hypochlorite or 80 ppm available free chlorine (AFC).

For daily dosing bd-tds.

Dispensed as irrigation solution or lavage.

d. Endophthalmitis

Intraocular infection is termed endophthalmitis, this can involve all layers of the eye and its contents. Introduction of intraocular microbes most commonly occurs during penetrating intraocular surgery, but can be secondary to trauma or spread of endogenous infection from remote sites via the blood stream.

The use of pre-operative topical antiseptics and post-operative antibiotics are critical steps in minimising endophthalmitis from skin and ocular commensals entering the eye during surgery. Endophthalmitis can result in loss of vision, inflammation of the eye, chronic pain and phthisis or shrinkage of the eye, sometimes requiring removal of the eye for pain relief.

e. Herpes Zoster Ophthalmicus and Other Viral Reactivations in and Around Eye and Periocular Skin.

### 2. Nasal Conditions

a. Rhinitis

Rhinitis is an inflammation of the nasal mucous membrane, which produces nasal congestion and rhinorrhea. It is typically of viral origin, but may involve secondary bacterial infection. Acute symptomatic rhinitis may be treated, for example, with decongestants such as pseudoephedrine. Where bacterial superinfection is involved, antibiotics may be administered.

Rhinorrhea and nasal congestion are typically of viral or allergic origin. Allergic congestion and rhinorrhea are treated with antihistamines. Topical or oral decongestants (e.g. pseudoephedrine) can provide some symptomatic relief, but prolonged use is not recommended.

b. Nasal Staphylococcus Trigger for Autoimmune Disease

Inflammatory conditions affecting the orbit and nasal cavity can be triggered by bacterial infection in the nasal cavity. Staphylococcus aureus is implicated in disease such as Granulomatosis with polyangiitis previously known as Wegener's granulomatosis, a rare systemic disorder that is associated with vasculitis (inflammation of the blood vessels). Typical signs and symptoms include nose bleeds, stuffy nose and nasal crustiness as well as orbital and ocular inflammation. Viruses and other microbes are also implicated in the pathology. Although treatment is with systemic immunosuppression, control of triggers such as nasal infection with topical and oral antibiotics is a main stay of treatment. Nasal infection is also implicated as a drive and trigger for Thyroid Eye Disease, or Graves' orbitopathy.

Composition and Delivery for Nasal Use:

Nasal pH compatible buffered hypochlorous solution with concentration of 0.008% sodium hypochlorite or 80 ppm available free chlorine (AFC). In many embodiments, the buffer may be useful in maintaining the appropriate pH. In some embodiments, the buffer is phosphoric acid.

For daily dosing bd-tds and long term use.

Dispensed as drop for nasal mucosa, or spray, or ointment with liquid paraffin base.

### 3. Ear Conditions

a. Otitis Media

Otitis media, inflammation of the middle ear structures, can lead to loss of balance and deafness. Otitis media is generally of bacterial or viral origin. Viral infections may compounded with secondary bacterial infections, including infections of Streptococcus pneumonia, Moraxella catarrhalis, and non typable Haemophilus influenzae. Where bacterial infection is suspected, the condition may be treated with antibiotics, as well as analgesia.

b. Otitis Externa

Ear infections, external otitis is an acute or chronic inflammation of the external ear canal, and may involve bacterial (e.g. Pseudomonas aeruginosa, Proteus vulgaris, and Staphylococcus aureus) or fungal (e.g. Aspergillus and Candida) infection. External otitis may be treated with, for example, antibiotics and corticosteroids.

c. Swimmer's Ear

Water retained in the ear canal after swimming can lead to inflammation and infection. Drying using alcohol has been used but can irritate and sting.

Composition and Delivery for auricular use:

Ear pH compatible buffered hypochlorous solution with concentration of 0.01% sodium hypochlorite or 100 ppm available free chlorine (AFC).

For daily dosing bd-tds and long term use.

Prevention of swimmer's ear with hypochlorous administration post-swimming using a dropper.

Dispensed as drop for external or middle ear, or spray.

### 4. Mouth Conditions

Pharyngitis (sore throat) is characterized by pain and swelling in the posterior pharynx. Pharyngitis is commonly caused by bacterial (e.g. Streptococcal) or viral infection, and may be treated with topical anaesthetics.

Stomatitis is a painful ulcer or inflammation of the oral mucosa. Stomatitis may be caused, for example, by infection (bacterial, viral, or fungal), chemical irritant, or allergic reaction.

Some common infectious agents include herpes simplex virus, varicella zoster, Epstein-Barr virus, influenza, cytomegalovirus, Gonorrhoea, and Candida. Treatments for stomatitis include anaesthetics, corticosteroids, antihistamines, and/or antibiotics.

Dental infections including post dental implants can result in breakdown of the mucosa and chronic non-healing mouth ulcers, these are managed with antibiotic and antibacterial mouthwash.

### Composition and Delivery for Oral Use:

Mouth pH compatible buffered hypochlorous solution with concentration of 0.008% sodium hypochlorite or 80 ppm available free chlorine (AFC).

For daily dosing bd-tds and long term use.

Dispensed as mouthwash/lavage or spray.

### 5. Genitourinary Conditions

### a) Vaginitis

Vaginitis or vulvovaginitis is inflammation of the vagina and vulva, and results in symptoms of itching, burning and discharge. Infections including bacteria, yeast and allergy from soaps or to spermicides can result in symptoms, and more than once cause can exist at a time. Treatment of infections is with topical or oral antibiotic or antifungals.

### b) STDs

A number of bacterial and viral infections can be transmitted between penis and vagina, mouth and vagina, mouth and penis, anus and mouth, anus and vagina, anus and penis. Hypochlorous spray before and after intercourse can help limit spread of viruses and bacteria.

### c) Urethritis

Urethritis is inflammation of the urethra, the tube that carries urine from the bladder to the outside body. It is commonly caused by infection due to bacteria, often from the skin around the urethra's opening. Bacteria that commonly cause urethritis include Gonococcus which is sexually transmitted and causes gonorrhoea, Chlamydia trachomatis also sexually transmitted and causes Chlamydia, and bacteria in and around stool and anus. Herpes simplex virus (HSV1 and HSV-2) can also cause urethritis. The sexually transmitted disease can result in chronic pelvic inflammatory disease in females and epididymitis in males, both leading to infertility. Treatment is with antibiotic or antivirals depending on the cause.

Vaginal pH (i.e. about 3.8 to 4.5) compatible buffered hypochlorous solution with concentration of 0.003% sodium hypochlorite or 80 ppm available free chlorine (AFC).

For daily dosing bd-tds and long term use.

Dispensed as spray or lavage and applied to affected areas, for example skin.

d. Balanitis

Balanitis is inflammation of the skin at the end of the penis (glans). This common condition can occur at any age, more commonly affecting boys under the age of 4 and males who are not circumcised. Bacterial commensals and candida are usually responsible as well as allergy. Treatment is with topical or oral antibiotic or anti-fungal and mild steroid cream.

### Composition and Delivery for Peri-Anal and Penile and Urethral Use:

Eye pH compatible buffered hypochlorous solution with concentration of 0.008% sodium hypochlorite or 80 ppm available free chlorine (AFC).

For daily dosing bd-tds and long term use.

Dispensed as spray or lavage.

### 6. Mastitis and Nipple Infection

Difficulty with breastfeeding and painful nipples are most often due to bacterial infection from Staphylococcus aureus and fungal infection by Candida this can result in infection of the breast tissue, mastitis, when bacteria enter a milk duct through a cracked nipple. Antibiotics and antifungals and anti-inflammatory medication are used for pain relief.

### Composition and Delivery for Breast Care:

Skin pH compatible buffered hypochlorous solution with concentration of 0.01% sodium hypochlorite or 100 ppm available free chlorine (AFC).

For daily dosing bd-tds and long term use.

Dispensed as spray/mist.

### 7. Skin

a. Eczema and infections in skin folds including in groin

b. Viral rash including shingles (varicella reactivation) including on skin and mucous membranes

c. Acne

d. Bacterial overgrowth in armpits and hidradenitis suppurativa

e. Post-operative promotion of healing, scar-reduction and prevention of infection

### Composition and Delivery for Skin Use:

Eye pH compatible buffered hypochlorous solution with concentration of 0.01-0.03% sodium hypochlorite or 100-300 ppm available free chlorine (AFC).

For daily dosing bd-tds and long term use.

Dispensed as spray or mist or solution.

### 8. Childcare

Prevention of nappy rash (diaper rash), prevention of infection spread from nipple to mouth, prevention and treatment of conjunctivitis including with delayed nasolacrimal duct canalisation. In these conditions, treatment may be, in some embodiments, via ointment or topical spray applied to the affected area, such as the skin.

9. Long-Term Care with Immunesuppression

Transplantation or autoimmune diseases are treated with immunosuppressants rendering the host more prone to infections. Regular use of hypochlorous may aid in reducing such infections.

### Examples

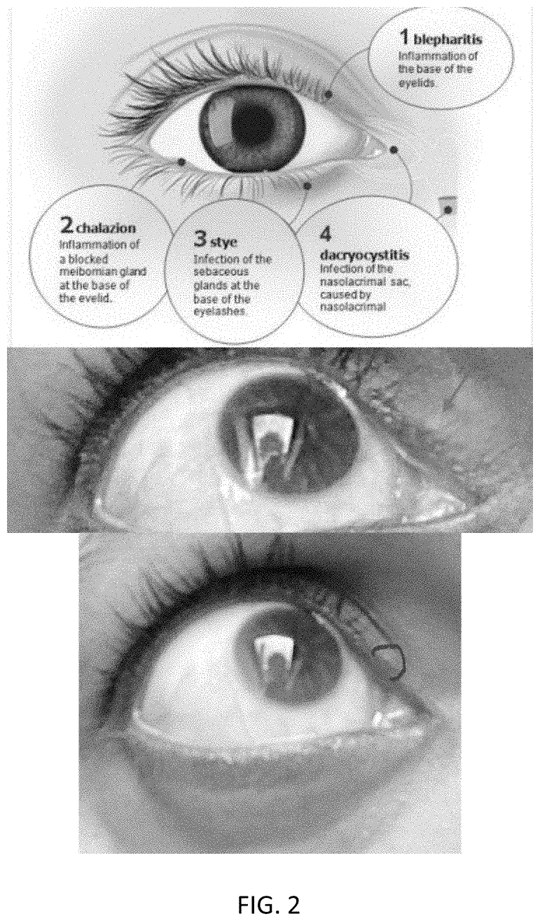

FIG. 2 shows results from treating a patient with an ocular condition for three days with one embodiment of the disclosed composition. The top panel of FIG. 2 is a diagram of the eye, showing various conditions that may be treated with the disclosed composition. The subject had a stye at the left upper eyelid. Subject was treated for three days with the disclosed composition.

FIG. 2, middle and lower panels show the subject's eye pre-treatment (middle panel; with arrow pointing to inflammation) and post-treatment (lower panel: resolved area partially circled). As FIG. 2 demonstrates, resolution of the stye was seen within 3 days of spraying (Purif Eyes™) spray. In many embodiments, resolution of symptoms (pain, redness, swelling, inflammation, etc.) may occur after about 2 days-6 weeks. In many embodiments, resolution may be in more than 1 day, 2 days, 3 days, 4 days, 5 days, 6 days, 7 days, 8 days, 9 days, 10 days, 2 weeks, 3 weeks, 4 weeks, 5 weeks, 6 weeks, or 7 weeks, and less than about 8 weeks, 7 weeks, 6 weeks, 5 weeks, 4 weeks, 3 weeks, 2 weeks, 11 days, 10 days, 9 days, 8 days, 7 days, 6 days, 5 days, 4 days, 3 days, or 2 days.

For mouth and eye conditions—clinical experience shows that the composition reduces inflammatory and infection signs and symptoms, within a matter of days or weeks, when used one, two, or three times a day directly on the eye surface or on mouth ulcers or hermetic skin lesions. Treated subjects have expressed surprise at the ease and simplicity of use, as well as the rapid resolution. For example:
This spray is amazing! I work on a dusty environment and find it so refreshing and easy to use to cleanse and protect
This was so easy to use after my eyelid surgery and when out and about there was no worry about mask wear and infection or using greasy ointment and eye drops in public. My eyelids healed very quickly
I sprayed this all over my eyes nose and mouth with the beginnings of a sore throat and I was better in less than 24 hours
Fantastic! Easier and more effective than eye drops. Great for when I've been wearing my contact lenses for too long and also for when I wake up with itchy eyes, before I put my glasses on. I've been using it a lot! Only downside is that I've had to switch to waterproof mascara because I also use it regularly on my eye lids
This product is easy to use and my husband says his eyes are more comfortable since using it
Gave it to my mom to treat her eyelids. They were red and sore and she could not wear contact lenses without her eyes watering. After 6 weeks she was much better and able to wear them again
I had a persistent stye (hordeolum) over a number of weeks, tried all old remedies but still no relief. Lucky enough to be recommended Purif by a friend, used the spray for two days as directed and stye completely disappeared! Amazing product which I can highly recommend as an addition to any medicine cabinet.

Clinical experience is that the product reduces inflammatory and infection signs and symptoms when used three times a day directly on the eye surface or on mouth ulcers or hermetic skin lesions within days.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A composition for treating a condition associated with a viral infection comprising:
a solvent;
hypochlorous, wherein the active free chlorine (AFC) of hypochlorous is between about 10 and 300 ppm, and the pH of the composition is between about 4 and 8.

2. The composition of claim 1, wherein the virus is SARS Coronavirus-19.

3. The composition of claim 2, wherein the pH is about 6.5 to 7.5.

4. The composition of claim 1, wherein the condition is selected from ocular, peri-ocular, nasal, ear, mouth, genitourinary, mastitis, nipple infection, eczema, viral rash, acne, hidradenitis suppurativa, wounds, incisions, diaper rash, blepharitis, canaliculitis and dacryocystitis, endophthalmitis, herpes zoster ophthalmicus, rhinitis, nasal staphylococcus, otitis media, otitis externa, swimmer's ear, pharyngitis, stomatitis, vaginitis, urethritis, balanitis, external and internal hordeolum, chalazion, and conjunctivitis.

5. The composition of claim 4, wherein the composition is a liquid, spray, ointment, or other topical treatment.

6. The composition of claim 5, wherein the AFC is from about 70 to 200 ppm.

7. A composition for treating a condition associated with an infection comprising:
a solvent;
hypochlorous, wherein the concentration of active free chlorine (AFC) is between about 10 and 300 ppm, and the pH of the composition is between about 4 and 8.

8. The composition of claim 7, wherein the infection is caused by bacterial, spores, fungi and viruses including, for example, without limitation, Apergillus niger, Candida albicans, Corynebacterium amycolatum, Enterobacter aerogenes, Escherichia coli, Haemophilus influenza, Klebsiella pneumoniae, Methicillin-resistant Staphylococcus aureus, Micrococcus luteus, Proteus mirabilis, Pseudomonas aeruginosa, Serratia marcescens, Staphylococcus aureus, Staphylococcus epidermidis, Staphylococcus haemolyticus, Staphylococcus hominis, Staphylococcus saprophyticus, Streptococcus pyogenes, Vancomycin-resistant Enterococcus faecium and viruses including Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae, for example SARS and MERS Coronavirus, low pathogenic avian influenza virus (AIV), H7N1.

9. The composition of claim 8, wherein the pH is about 6.5 to 7.5.

10. The composition of claim 7, wherein the condition is selected from ocular, peri-ocular, nasal, ear, mouth, genitourinary, mastitis, nipple infection, eczema, viral rash, acne, hidradenitis suppurativa, wounds, incisions, diaper rash, blepharitis, canaliculitis and dacryocystitis, endophthalmitis, herpes zoster ophthalmicus, rhinitis, nasal staphylococcus, otitis media, otitis externa, swimmer's ear, pharyngitis, stomatitis, vaginitis, urethritis, balanitis, external and internal hordeolum, chalazion, and conjunctivitis.

11. The composition of claim 9, wherein the composition is a liquid, spray, ointment, or other topical treatment.

12. The composition of claim 9, wherein the AFC is from about 70 to 200 ppm.

13. The composition of claim 8, wherein the virus is SARS Coronavirus-19.

14. A method of treating or preventing a viral infection comprising:
dispensing a sufficient amount of a composition comprising hypochlorous toward an eyeball of a subject;
allowing the composition to contact the eyeball; thereby
treating or preventing a viral infection.

15. The method of claim 14, wherein the concentration of active free chlorine (AFC) in the composition is between about 10 and 300 ppm.

16. The method of claim 15, wherein the virus is selected from Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae, for example SARS and MERS Coronavirus, low pathogenic avian influenza virus (AIV), H7N1, and COVID-19.

17. The method of claim 16, wherein the pH is about 4 to 8.

18. The method of claim 16, wherein the viral agent is SARS Coronavirus-19.

19. The method of claim 17, wherein the concentration of AFC in the composition is between about 70 and 200 ppm, and the pH of the composition is between about 4 and 8.

20. The method of claim 19, wherein the concentration of AFC is about 80 ppm and the pH of the composition is about 6.5 to 7.6.
